(19) United States
(12) Patent Application Publication
(10) Pub. No.: US 2016/0089080 A1
(43) Pub. Date: Mar. 31, 2016

(54) SYSTEM AND METHOD FOR ACTIVITY DETERMINATION

(71) Applicant: mophie, Inc., Tustin, CA (US)

(72) Inventors: Fang Li, Milbrae, CA (US); Zhihao Chen, Irvine, CA (US); Cheng Cao, Tustin, CA (US)

(21) Appl. No.: 14/732,438

(22) Filed: Jun. 5, 2015

Related U.S. Application Data
(60) Provisional application No. 62/057,931, filed on Sep. 30, 2014.

(51) Int. Cl. A61B 5/00; G01P 15/00

(52) U.S. Cl. CPC A61B 5/6801; G01P 15/00

(57) ABSTRACT

Features are disclosed relating to determining an activity in which a user is (or has been) engaged. One such activity is the taking of steps (e.g., walking or running). Some embodiments described herein are directed to accurate detection and counting of steps made by a user wearing a device with step-detection functionality. The accurate step counting can be facilitated by detecting signatures of certain activities, and determining whether to count steps based on an analysis of acceleration data over various intervals and moving windows of time.

### CROSS-REFERENCE TO RELATED APPLICATION

This application claims the benefit of U.S. Provisional Patent Application No. 62/057,931, filed on Sep. 30, 2014 and entitled “SYSTEM AND METHOD FOR ACTIVITY DETERMINATION,” the entirety of which is incorporated by reference herein.

### BACKGROUND

Pedometers and other step-counting devices enable measurement of the number of steps made by a user. Many step-counting devices also calculate of the distance traveled and supply other information, such as average speed, calorie consumption, etc. Pedometers are used in a wide range of applications in the clinical sector (e.g., in rehabilitation), and in general in the field of fitness (e.g., as instruments for monitoring a physical activity). Some pedometers use integrated accelerometers (e.g., micro-electromechanical systems or “MEMS”) for step detection. Such pedometers have particularly compact dimensions, and can be integrated within portable electronic devices such as mobile phones, media players, wearable devices, and the like.

Step-counting devices typically implement a step detection method based upon an analysis the vertical acceleration pattern generated during the various phases of the step (e.g., by the contact of a user's foot with the ground, by lift-off of a foot from the ground, etc.). The acceleration data used by such step detection methods is provided by an accelerometer fixed to the body of the user, typically in the form of a pedometer or other step-counting device worn on the wrist or belt. Generally described, vertical acceleration refers to an acceleration directed along the vertical axis of the user's body. In particular, the occurrence of a step is determined by identifying acceleration peaks that appear in the acceleration signal. The peaks are detected by comparing the acceleration signal with a given reference threshold having a pre-set value.

### SUMMARY OF CERTAIN EMBODIMENTS

The summary of certain embodiments provided herein is not intended to be limiting and provides some examples of the various embodiments disclosed herein. Various alternatives and other embodiments are disclosed herein.

Various embodiments disclosed herein relate to a device that can include one or more sensors configured to detect acceleration, and one or more physical computer processors configured to at least obtain acceleration data from the one or more sensors for a time interval, determine that the acceleration data is not indicative of a non-step-related activity, average the acceleration data for the time interval to obtain a mean acceleration vector, project the mean acceleration vector on a gravity acceleration vector to obtain a projection, identify a peak using a derivative based at least partly on the projection, and determine that a user of the device has taken a step based at least partly on the identified peak.

The one or more computer processors can be configured to determine that the acceleration data is not indicative of a non-step-related activity by extracting one or more features from the acceleration data and analyzing the features to determine whether they correspond to features of data associated with a non-step-related activity. The non-step-related activity can be, for example, driving, eating, brushing teeth, or sleeping. The one or more computer processors can be configured to determine that the acceleration data is not indicative of a non-step-related activity by analyzing the acceleration data with respect to data associated with mechanical vibrations.

The one or more computer processors can be configured to determine whether a number of peaks identified in a window of time exceeds a threshold.

The one or more sensors can include a 3-axis accelerometer. The one or more processors can be configured to obtain data from a plurality of sensors. The plurality of sensors can include an accelerometer and a gyroscope.

The one or more processors can be configured to at least identify a plurality of steps during a time window, determine that the user is performing a stepping activity when the plurality of steps during the time window are above a threshold number, and determine that the user is not performing a stepping activity when the plurality of steps during the time window are not above the threshold number.

Various embodiment disclosed herein can relate to non-transitory computer-readable storage storing executable instructions that cause one or more computer processors to perform a process that includes obtaining acceleration data for a first time interval, determining that the acceleration data is indicative of an active state of a user, averaging the acceleration data for the first time interval to obtain a mean acceleration vector, projecting the mean acceleration vector on a gravity acceleration vector to obtain a projection, identifying a peak using a derivative based at least partly on the projection, and determining that the user has taken a step based at least partly on the identified peak.

Determining that the acceleration data is indicative of an active state of a user can include using a classification model trained to detect a signature of user activity in acceleration data.

The process can include obtaining acceleration data for a second time interval, determining that the acceleration data is indicative of an inactive state of a user, and changing a rate at which acceleration data is sampled for a third time interval. The process can include obtaining acceleration data for the third time interval, determining that a movement magnitude represented by the acceleration data fails to exceed an idle state threshold, and determining that the user is sleeping based at least partly on the determination that the movement magnitude fails to exceed the idle state threshold. The process can include obtaining acceleration data for the third time interval, determining that a movement magnitude represented by the acceleration data exceeds an idle state threshold, and determining that the user is not sleeping based at least partly on the determination that the movement magnitude exceeds the idle state threshold. The process further can include obtaining acceleration data for the third time interval, determining that a movement magnitude represented by the acceleration data fails to exceed a sleeping state threshold, and determining that the acceleration for the third time interval is indicative of non-use.

Various embodiment disclosed herein can relate to a system that includes one or more sensors configured to detect acceleration, and one or more computer processors configured to at least obtain acceleration data from the one or more sensors for a first time interval, determine that the acceleration data for the first time interval is indicative of an active state of a user, obtain acceleration data from the one or more sensors for a second time interval, determine that the acceleration data for the second time interval is indicative of an inactive state of a user, and change a rate at which acceleration data is sampled for a third time interval.

The one or more computer processors can be configured to determine that the acceleration data is indicative of an active state of a user comprises using a classification model trained to detect a signature of user activity in acceleration data.

The one or more computer processors can be configured to at least average the acceleration data for the first time interval to obtain a mean acceleration vector, project the mean acceleration vector on a gravity acceleration vector to obtain a projection, identify a peak using a derivative based at least partly on the projection, and determine that the user has taken a step based at least partly on the identified peak.

The one or more computer processors can be configured to at least obtain acceleration data for the third time interval, determine that a movement magnitude represented by the acceleration data fails to exceed an idle state threshold, and determine that the user is sleeping based at least partly on the determination that the movement magnitude fails to exceed the idle state threshold. The one or more computer processors can be further configured to at least obtain acceleration data for the third time interval, determine that a movement magnitude represented by the acceleration data exceeds an idle state threshold, and determine that the user is not sleeping based at least partly on the determination that the movement magnitude exceeds the idle state threshold. The one or more computer processors can be configured to at least obtain acceleration data for the third time interval, determine that a movement magnitude represented by the acceleration data fails to exceed a sleeping state threshold, and determine that the acceleration for the third time interval is indicative of non-use.

The one or more processors can be configured to at least identify a plurality of steps during a time window, determine that the user is performing a stepping activity when the plurality of steps during the time window are above a threshold number, and determine that the user is not performing a stepping activity when the plurality of steps during the time window are not above the threshold number.

### BRIEF DESCRIPTION OF DRAWINGS

Embodiments of various inventive features will now be described with reference to the following drawings. Throughout the drawings, reference numbers may be re-used to indicate correspondence between referenced elements. The drawings are provided to illustrate example embodiments described herein and are not intended to limit the scope of the disclosure.

FIG. 1 shows a user taking several steps while wearing an illustrative step-counting device.

FIG. 2 is a block diagram of an illustrative activity determination device (e.g., a step-counting device) showing various components.

FIG. 3 is a flow diagram of an illustrative process for determining an activity of a user according to some embodiments.

FIGS. 4A and 4B are flow diagrams of an illustrative process for counting steps according to some embodiments.

FIG. 5 shows graphs of data captured during a non-step-related activity and a step-related activity.

FIG. 6 is a diagram of average acceleration vectors over successive time intervals.

FIG. 7 is a diagram of an illustrative angle, in 3-dimensional space, between average acceleration vectors over successive time intervals.

FIG. 8 is a diagram of an illustrative sliding window of acceleration measurements over a period of time.

### DETAILED DESCRIPTION OF EMBODIMENTS

### Introduction

Various embodiments of the present disclosure are directed to determining an activity in which a user is (or has been) engaged. One such activity is the taking of steps (e.g., walking or running). Some embodiments described herein are directed to accurate detection and counting of steps made by a user wearing a device with step-detection functionality. The accurate step counting can be facilitated by detecting signatures of certain activities, and determining whether to count steps based on an analysis of acceleration data over various intervals and moving windows of time.

Many conventional step-counting devices use 2-axis accelerometer data to detect and count steps. Peaks in the data exceeding some threshold can trigger detection and counting of a step. However, such methods may produce inaccurate results by detecting and counting steps in situations in which the wearer of the conventional step counting device is not actually walking or running. For example, some conventional step-counting devices may count steps when the user is actually driving, brushing his/her teeth, shaking hands, eating, or the like. Such inaccurate results may be caused by the use of 2-axis accelerometer data, which does not provide information about acceleration in 3-dimensional space. Therefore, some movements (e.g., brushing teeth) may cause the accelerometer to produce data with properties (e.g., peaks) similar to data produced when a user is walking or running. In addition, conventional step-counting devices may produce inaccurate results by detecting and counting individual steps or small groups of steps in periods of time which do not correspond to how users typically walk and/or run. For example, some conventional step-counting devices may count one or two steps over a relatively long period of time (e.g., several seconds or minutes) even though the data used to detect such steps was produced by other non-step-based movements, such as the examples given above. Over the course of day, such false step detections can add up to a significant number of false steps (e.g., hundreds of additional steps).

Some aspects of the present disclosure relate to analyzing acceleration data in 3-dimensional space. By analyzing acceleration data in 3-dimensional space (e.g., as provided by a 3-axis accelerometer, an accelerometer and a gyroscope in combination, etc.), signatures of non-step-related data can be identified even in situations where data from a t-axis accelerometer is easily confusable for step-related data. For example, a user may wear a step counting device while driving, and the step counting device may generate or obtain acceleration data. The step counting device can identify a non-step-related signature in the data, such as a data signature indicative of driving or unnatural motion. In such cases, the step counting device can exclude or suppress any step detections made using such data. In some embodiments, the data may be analyzed for signatures of non-step-related data, and the step counting analysis may not be performed at all on such data.

Additional aspects of the present disclosure relate to analyzing acceleration data for various time intervals and moving windows of time. By averaging data over a time interval, the effect of individual anomalous samples (or small groups thereof) can be offset by the rest of the data obtained for the interval. Thus, step detections based on anomalous samples can be reduced or eliminated. In addition, by analyzing data over moving windows of time, individual steps (or small groups thereof) detected in a window of time can be excluded when such detections are likely to be anomalous. For example, users typically take steps in groups, particularly when walking or running for exercise. By waiting to count steps until a threshold number of steps are detected in a window of time, anomalous individual step detections can be suppressed. When the number of steps detected in a window of time exceeds a threshold, those steps can be retroactively added to the total step count.

Although the examples and embodiments described herein will focus, for the purpose of illustration, on detecting signatures of specific non-step-related activities, one skilled in the art will appreciate that the techniques described herein may be applied to detecting signatures of any non-step-related activity. In addition, although the examples and embodiments described herein will focus, for the purpose of illustration, on analyzing acceleration data over particular intervals and windows of time, one skilled in the art will appreciate that the techniques described herein may be applied to any interval or window of time. Furthermore, although the examples and embodiments described herein will focus, for the purpose of illustration, on step-counting devices worn on a user's wrist, one skilled in the art will appreciate that the techniques described herein may be applied to other step counting devices, such as those worn on a belt, ankle or shoe, those carried in a user's hand or pocket, those integrated with or attached to headwear, etc. Various aspects of the disclosure will now be described with regard to certain examples and embodiments, which are intended to illustrate but not limit the disclosure.

Example Activity Determination Device

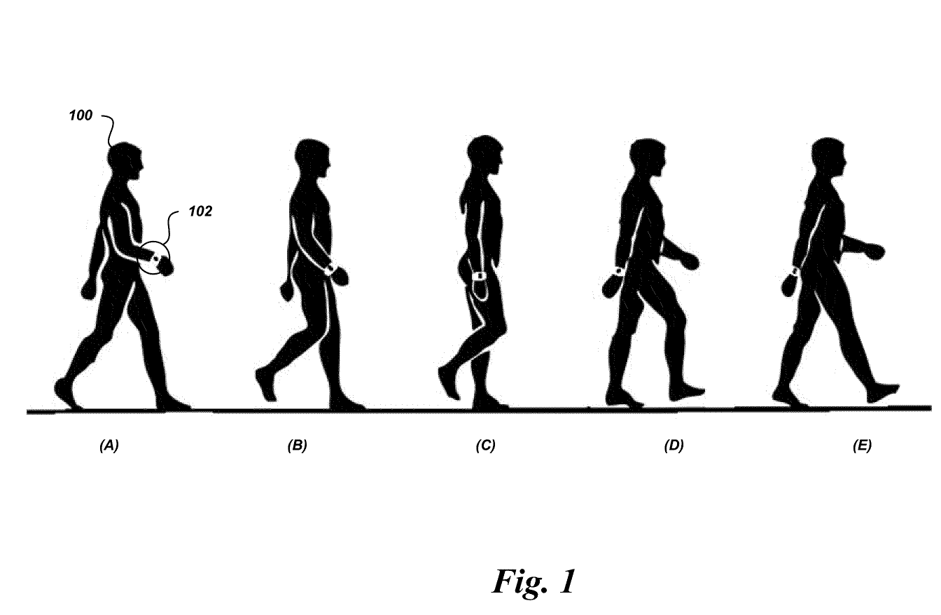

FIG. 1 illustrates an example user 100 walking over several successive points in time (A)-(E) while wearing an activity determination device 102 (e.g., a step counting device). As the user 100 takes steps, whether walking or running, he lifts his rear foot off the ground and places it back in contact with the ground at a point in front of the opposite foot. The process is then repeated with the other foot as the user continues to walk or run. Each lift-off and replacement of a foot corresponds to a single step. During the process of taking a step, the user 100 typically swings one arm from behind or beside the user to a point in front of the user, while simultaneously swinging the opposite arm from in front of the user to a point behind or beside the user. In addition, as the user's body moves up and down during the stepping process (due to the lift off and replacement of the user's feet), the user's arms also move up and down.

A step counting device 102 worn on the user's arm can detect the user's movements throughout the step process by monitoring the acceleration experienced by the step counting device 102 at various points in time. For example, as shown, the user 100 may wear the step counting device 102 on the user's right arm. At time (A), the user's right arm (and, therefore, the step-counting device 102) may be in front of the user 100, and the user's left arm may be behind the user 100. As the user 100 continues to step forward, the right arm moves backward and the left arm moves forward until, at time (E), the user's arms have switched positions (along with the user's feet). This pendulum-like movement generally continues during the time in which the user is walking, with the user taking a full step with one foot each time the user's arms switch positions from front to back. The step counting device 102 can monitor the acceleration caused by up-and-down and/or back-and-forth movements of the user's arms by repeatedly recording acceleration measurements at various points in time. As described herein, the step counting device 102 can then use those measurements to accurately detect and count steps taken by the user 100 while also reducing or eliminating false step detections based on acceleration data attributable to non-step-related activity, such as driving, brushing teeth, eating, and the like.

The activity determination device 102 (e.g., the step counting device) may be implemented as a variety of mobile electronic devices. For example, the activity determination device 102 may be any wearable device (e.g., “fitness trackers,” “smart watches,” “smart eyewear”), hand held computing device (e.g., mobile phones or tablet computing devices), or some other mobile electronic device or appliance. The activity determination device 102 may include various sensors, processors, and storage for detecting movements, analyzing the movements to determine an activity of the wearer, analyzing the movements to detect steps, and storing information about wearer activity and/or detected steps.

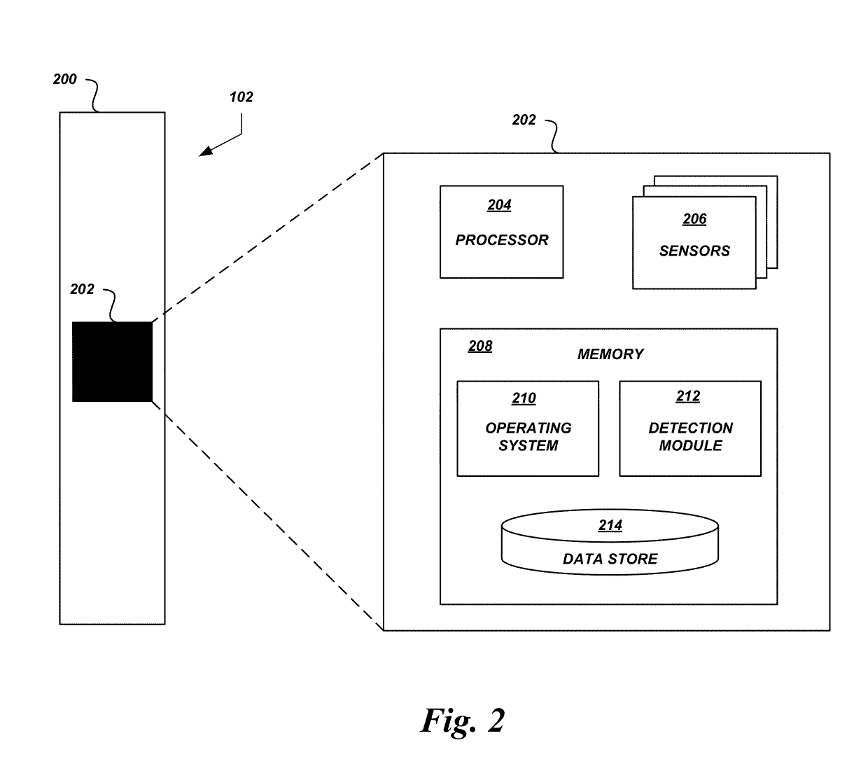

In some embodiments, the activity determination device (e.g., step counting device) 102 may include an attachment structure to attach the device 102 securely to the user 100 (e.g., to the user's arm, clothing, etc.), and a housing for various electronic and/or mechanical components that detect and count steps and/or determine user activity. For example, as shown in FIG. 2, the activity determination device 102 may include a wrist band 200 and a housing 202. The housing 202 may include various components, including but not limited to: one or more electronic computer processors 204 configured to analyze data regarding movements of the activity determination (e.g., step counting) device 102 (and, therefore, the user 100) and to otherwise manage operation of the device 102; one or more sensors 206, such as accelerometers and/or gyroscopes, configured to provide data regarding movements of and acceleration experienced by the device 102; and a memory 208 configured to store data and computer-executable instructions for providing the step counting and activity determination features described herein. Illustratively, the memory 208 may store an operating system 210 to facilitate management of the activity determination device (e.g., step counting device) 102, a detection module 212 that includes instructions for one or more activity determination, step-detection, and/or step counting algorithms, and a data store 214 to store activity information, counted steps, and/or other operational data.

The example modules and components shown in FIG. 2 and described herein are illustrative only, and are not intended to be limiting. In some embodiments, the activity determination (e.g., step counting) device 102 may include additional, fewer, or alternative components. For example, the activity determination (e.g., step counting) device 102 may include a hardware-based detection module 212 implemented as an application-specific integrated circuit (ASIC) rather than a software module of executable instructions stored in a computer memory. As another example, the activity determination (e.g., step counting) device 102 may include various electronic components to facilitate operation of the modules and components shown, such as buses, communication interfaces and ports, user interfaces, batteries and other power sources, and the like.

Example Processes for Activity Determination (e.g., Detecting and Counting Steps)

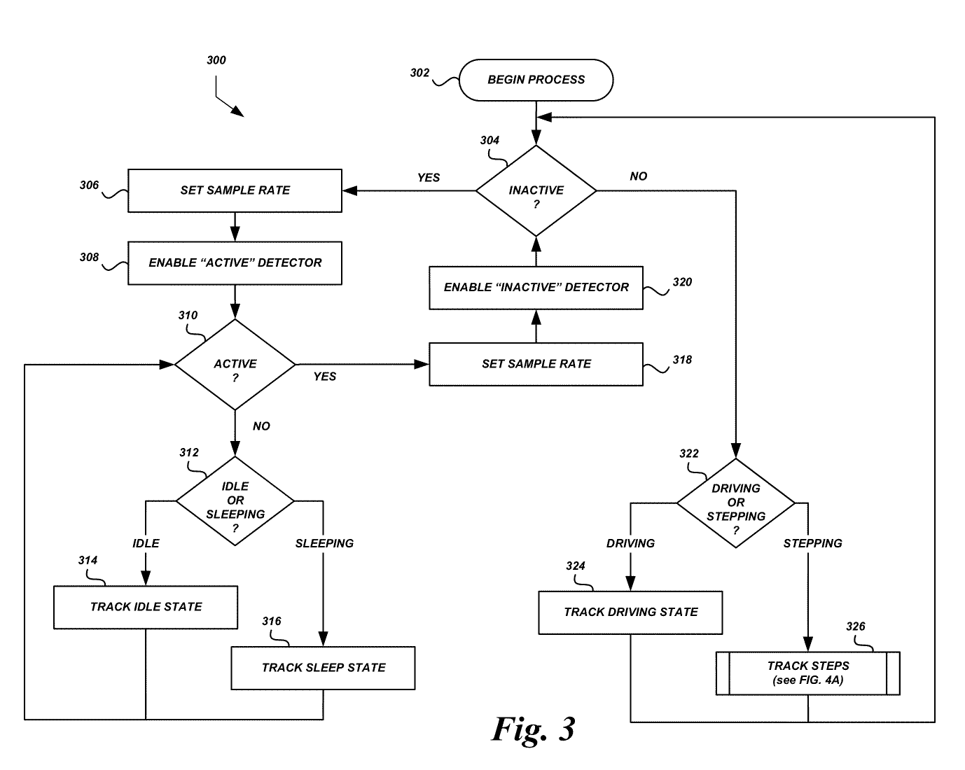

FIG. 3 depicts an illustrative process 300 for determining activities in which a user is (or has been) engaged. For example, the process 300 may determine whether a user is active or inactive. When inactive, the process can advantageously switch to a lower-power mode with a reduced input data sample rate in order to conserve battery life. In addition, when the user is inactive, the process can determine whether a user is sleeping vs. merely idle, while continuing to monitor for a time at which the user becomes active again. Although the process 300 is described below with respect to a step counting device 102, the process 300 may be partially or wholly implemented by some other activity determination or detection device.

The process 300 may be embodied in a set of executable program instructions stored on a computer-readable medium, such as a detection module 206 stored in memory 208 of the step counting device 102 (or some other activity determination device). When the process 300 is initiated at block, 302, the executable program instructions can be accessed and executed by one or more processors 204 of the step counting device 102.

At decision block 304, the step counting device 102 can determine whether a user is inactive (e.g., the user is not currently walking or running, driving a car, etc.). One or more sensors 206 may provide data, and a classifier or some other model can be used to analyze the data and determine whether it is indicative of inactivity. In some embodiments, the data may include 3-axis acceleration data from a 3-axis accelerometer. In other embodiments, the data may include 3-axis data from a gyroscope and a 2- or 3-axis accelerometer. The data may be sampled at a single constant rate (e.g., about 25 Hz), or the data may be sampled at two or more different rates, depending on a configuration setting or dynamically-determined criteria. For example, data may be sampled at a default rate (e.g., about 25 Hz) and a reduced rate (e.g., about 6 Hz). The default rate may be used when the process 300 is initiated and during periods of activity (e.g., when the user is not sleeping, when the number of detected steps for a window of time meets or exceeds a threshold, etc.). The reduced rate may be used during periods of inactivity (e.g., when a user is detected as sleeping, when the number of detected steps for a window of time has not met a threshold, etc.). Such reduced sample rates can extend the battery life of the step counting device 102.

An “inactive” state detector may be used at decision block 304 to determine whether the user is currently inactive (e.g., sleeping or otherwise idle). The “inactive” state detector may be or use a model that is trained to classify input data as indicative of inactivity. In response to such a classification, or to a threshold number or percentage of such classifications in a period of time, the process 300 may proceed to block 306. Otherwise, if the “inactive” state detector does not detect inactivity, the process 300 may proceed to decision block 322. In some embodiments, the detector used at decision block 304 may be an “active” state detector that detects whether a user is currently active (e.g., the user is walking, running, driving a car, or is otherwise not idle), rather than (or in addition to) an “inactive” state detector.

At block 306, the step counting device 102 can change to a lower data sample rate, such as the reduced sample rate described above, to extend the battery life of the step counting device 102 while tracking the user's inactive state.

At block 308, the step counting device 102 can enable an “active” state detector that detects whether a user is currently active (e.g., the user is walking, running, driving a car, or is otherwise not idle). The active state detector enabled at block 308 may be a separate detector (e.g., may use a different model) than the active or inactive state detector used in decision block 304 due to the different sample rates. For example, the active state detector enabled or activated at block 308 may use a model that is trained to use data sampled at the reduced sample rate set above at block 306, rather than the default sample rate. In some embodiments, the detector enabled at block 308 may be an inactive state detector that detects whether a user is currently inactive (e.g., the user is not walking, running, driving a car, etc.), rather than (or in addition to) an active state detector.

At decision block 310, the step counting device 102 can determine whether the user is currently active (e.g., by using the active state detector enabled above). If so, the process 300 can proceed to block 318, where the sample rate may be reset to the default sample rate, and then to block 320, where the inactive state detector is enabled (and the active state detector is disabled, deactivated, suppressed, etc.). Otherwise, if the active state detector determines that the user is not active, the process 300 can proceed to decision block 312.

At decision block 312, the step counting device 102 can determine whether the user is sleeping or merely idle. The determination may be made using one or more classifiers or other models trained to detect the signature of sleep or mere idleness in input data. For example, an analysis of the frequency and magnitude of user movements may be indicative of mere idleness if the frequency exceeds a threshold and/or the magnitude (e.g., the amplitude of accelerometer data that indicates changes in acceleration) exceeds a threshold while still being identified as inactive (above at block 310). In such cases, the process 300 may proceed to block 314, where the step counting device 102 tracks the idle state of the user (e.g., starts or continues a timer, increments a counter, etc.). The analysis at block 312 may be indicative of sleep if the frequency of movement falls below a threshold and/or the magnitude (e.g., the amplitude of accelerometer data) falls below a threshold. In such cases, the process 300 may proceed to block 316, where the step counting device 102 tracks the sleep state of the user (e.g., starts or continues a timer, increments a counter, etc.).

In some embodiments, the step counting device 102 can distinguish sleep from non-use of the step counting device 102. For example, if a user takes off the step counting device 102 and places it somewhere stationary (e.g., a dresser), the device 102 may not detect any movement at all for a threshold period of time. In contrast, users typically move even when sleeping, such as subtle movements caused by breathing and detected by the step counting device 102. When the frequency and/or magnitude of user movements fall below a threshold for mere idleness but exceed a threshold indicative of non-use, then the user may be determined to be sleeping. Otherwise, if the frequency and/or magnitude of detected movements fall below a threshold for sleeping, then the step counting device 102 may be determined to be not in use.

The active state detector may remain active for each item or group of incoming data, and the process 300 may make a continuous loop to block 310 to determine whether the user is active or is still inactive. If the user is determined to be active, the process 300 can proceed to block 318, as described above. In some embodiments, the determination described herein with respect to block 310 may be performed in parallel with the determination described with respect to block 312.

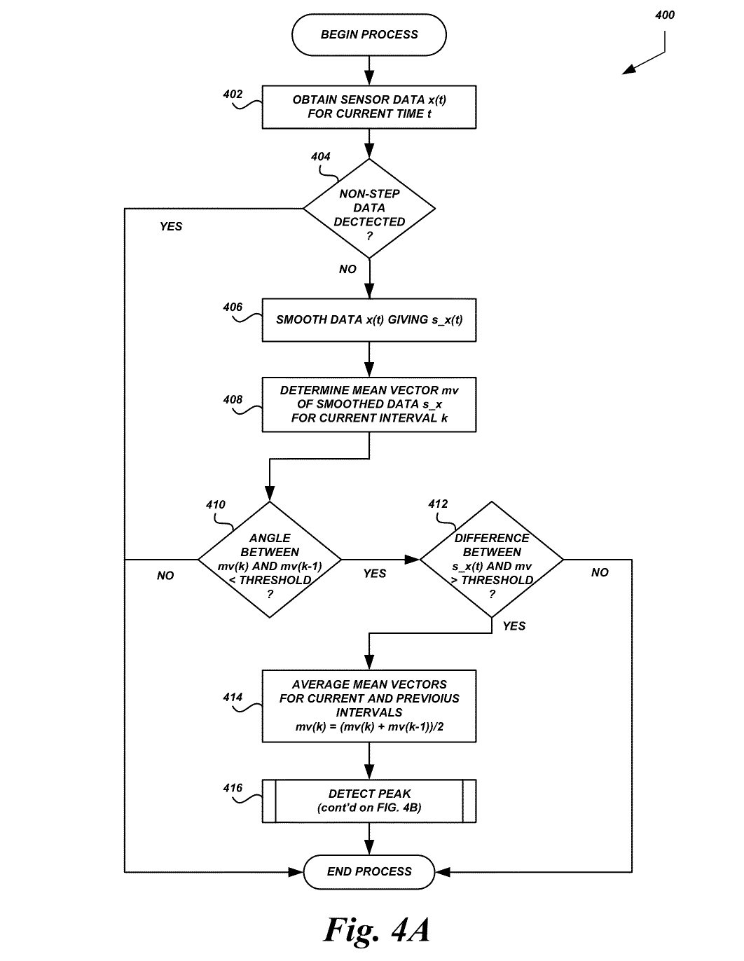

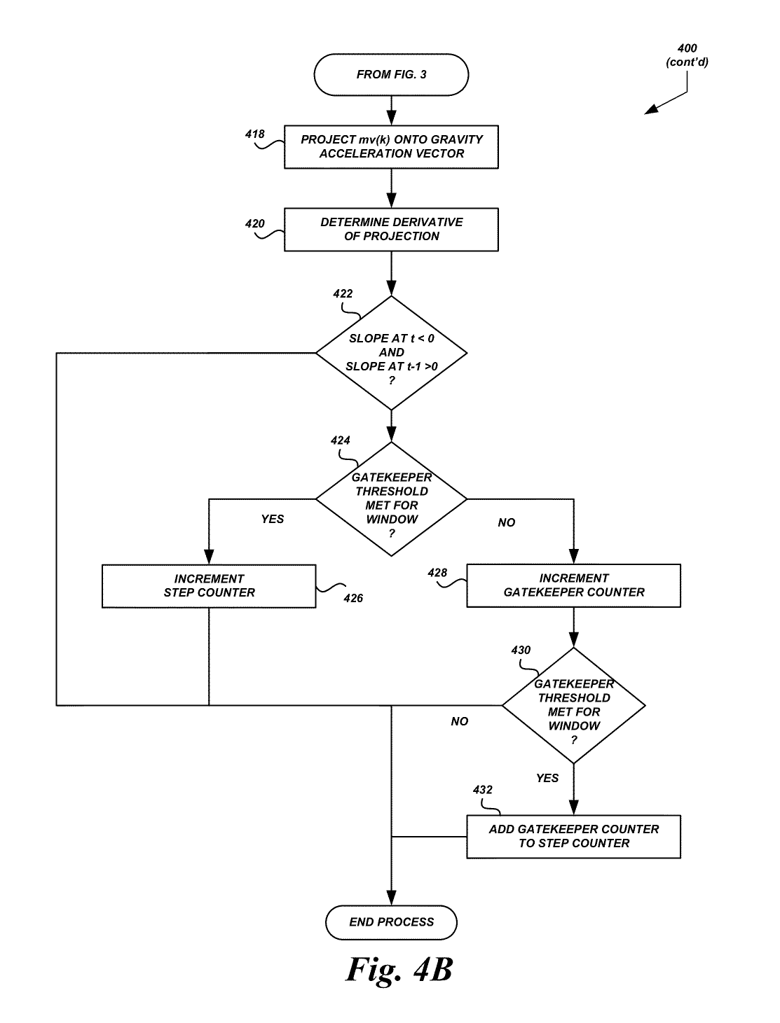

At decision block 322, in response to the step counting device 102 determining that the user is not inactive (or determining that the user is in fact active), the step counting device 102 can determine whether the user is driving or is stepping (e.g., walking or running) or otherwise physically active. The determination may be made using one or more classifiers or other models trained to detect the signature of driving or stepping in input data. For example, an analysis of the frequency and magnitude of user movements may be indicative of driving if a mechanical and repetitive frequency is detected. In such cases, the process 300 may proceed to block 324, where the step counting device 102 tracks the driving state of the user (e.g., starts or continues a timer, increments a counter, etc.). The analysis at block 322 may be indicative of stepping or some other physical activity if mechanical and repetitive frequency not reasonably caused by human movement is detected. In such cases, the process 300 may proceed to block 326, where the step counting device 102 tracks the steps of the user. In some embodiments, the device 102 can distinguish between running steps and walking steps (which can be tracked separately or together). An example process for tracking steps is shown in FIGS. 4A and 4B and described in greater detail below. In some embodiments, the decision block 322 can be used to determine various other types of activities, other than stepping or driving, such as brushing teeth, eating, etc. In some embodiments, the decision block 322 can make a determination that the user is engaging in an unspecified activity in the active state, for example, if the data collected in the active state does not fit any of the specific activities that the device 102 is configured to identify.

The inactive state detector may remain active for each item or group of incoming data, and the process 300 may make a continuous loop to block 304 to determine whether the user is inactive or is still active. If the user is determined to be inactive, the process 300 can proceed to block 306, as described above. In some embodiments, the determination described herein with respect to block 304 may be performed in parallel with the determination described with respect to block 322.

FIGS. 4A and 4B depict an illustrative process 400 for detecting and counting steps. Advantageously, the process 400 may determine when input data is indicative of various non-step-related activities, and suppress any steps counted using such data or otherwise exclude such data from the step counting process. In addition, the process 400 may use various time intervals and sliding windows to reduce the number of false positives (e.g., steps erroneously detected and counted based on anomalous data).

The process 400 may be embodied in a set of executable program instructions stored on a computer-readable medium, such as a detection module 206 stored in memory 208 of the step counting device 102. When the process 400 is initiated, the executable program instructions can be accessed and executed by one or more processors 204 of the step counting device 102. The process 400 may be executed separately and sequentially for each sample of data provided by one or more of the sensors 206. In some embodiments, the process 400 (or portions thereof) may be executed in parallel for multiple samples of data provided by the sensors 206. In additional embodiments, the process 400 (or portions thereof) may be executed on a group of multiple data samples (e.g., a “batch” of data).

At block 402, one or more sensors 206 may provide data that can be used to detect steps taken by the user 100 of the step counting device 102. In some embodiments, as described above with respect to the process 300, the data may include 3-axis acceleration data from a 3-axis accelerometer, or the data may include 3-axis data from a gyroscope and a 2- or 3-axis accelerometer the data may be sampled at a single constant rate (e.g., about 25 Hz), or the data may be sampled at two or more different rates, depending on a configuration setting or dynamically-determined criteria, etc. For example, data may be sampled at a first lower rate (e.g., about 6 Hz, or between about 4 Hz and about 10 Hz, or between about 2 Hz and about 15 Hz) during period of inactivity (e.g., when a user is detected as sleeping or idle, or when the number of detected steps for a window of time has not met a threshold, etc.). The data may then be sampled at a second higher rate (e.g., about 25 Hz, or between about 20 Hz and about 30 Hz, or between about 15 Hz and about 35, 40, 45, 50, or 60 Hz) during periods of activity (e.g., when the user is not sleeping, when the number of detected steps for a window of time meets or exceeds a threshold, etc.). Reduced sample rates can extend the battery life of the step counting device 102. As shown and described herein, the sensor data may be denoted x(t), where t is the time instance or period to which the sensor data corresponds.

At decision block 404, the step counting device 102 can determine whether the input data is indicative of a particular non-step-related activity or is otherwise not indicative of step-related activity. For example, the step counting device 102 may have access to one or more signatures or models of data corresponding to a non-step-related activity (e.g., brushing teeth, sleeping) and/or one or more models of data that is not indicative of step-related activity (e.g., mechanical vibrations that occur during driving). The step counting device 102 may compare the current input data (and, in some cases, an amount of previously-received input data, such as data for some predetermined or dynamically determined period of time), or some data derived therefrom, to the signature(s). If the current data approximates a model of non-step-related activity (e.g., a plot of input data is a “good fit” for a plot of non-step-related data, or statistical features of the input data correspond sufficiently to statistical features of non-step-related data), then the process 400 may terminate or otherwise suppress counting a step using the current data.

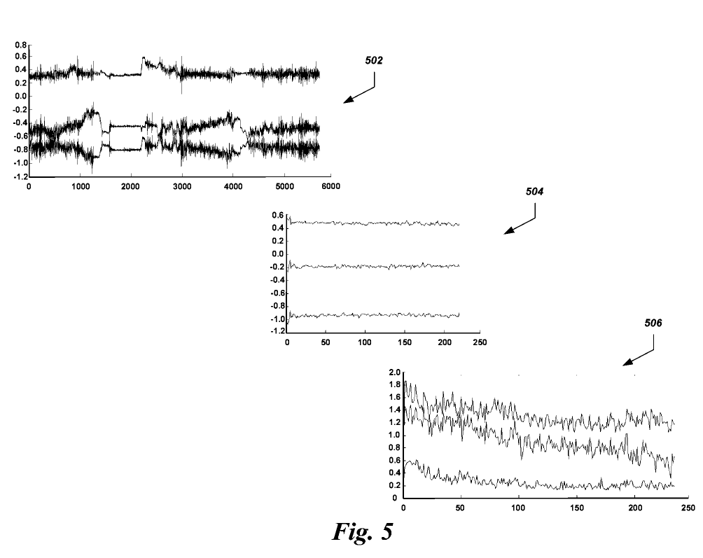

In some embodiments, the step counting device 102 can compute amplitude, frequency and/or other features of the input data for some window of time (e.g., about 3 seconds, 2 seconds, 1 second, 0.5 seconds, 0.1 seconds, etc.). The features of the current input data can be analyzed using models of non-step-related activity, mechanical vibrations, or the like to determine whether the input data likely corresponds to non-step-related activity (or mechanical vibrations, etc.). FIG. 5 shows an example graph 502 of 3-axis data corresponding to driving. FIG. 5 also shows example 3-axis data graphs for walking 504 and running 506, respectively. As shown, the step-related walking data 504 and running data 506 are different in amplitude and/or frequency than the non-step-related driving data 502. Input sampled during periods of walking or running (such as the sample walking data 504 or running data 506 shown) will not sufficiently correspond to the driving data 502, and the process 400 can proceed to block 406.

In some embodiments, statistical features extracted from input data can be analyzed using a machine learning classification or regression model. For example, a machine learning model may be trained to classify or predict whether input data corresponds to a particular non-step-related activity, such as driving. Features used by the machine learning model may include: the standard deviation of the normal distribution (“norm”) of the acceleration; the frequency of the vibration of the norm of the acceleration (e.g., estimated by the occurrences of the acceleration norm jump from below the average to above the average); the norm of the linear slope of the acceleration on each axis (e.g., where the definition of slope of each axis acceleration is given by a linear regression model); the average of the standard deviations of the acceleration on each axis; the effectiveness of the linear regression model(s) defining the slope of each axis of acceleration; and/or the average of the segmental-variation of the acceleration on each axis.

Illustratively, the definition of the slope of the acceleration on each axis may be determined using the linear regression model set forth in Equation [1]:

aᵢ(k)=slopeᵢ*t(k)+nᵢ(k)+mᵢ [1]

where i=1, 2, 3; k=1, 2, . . . 10*sampling rate (e.g., 25 Hz); and t(k)=k/sampling rate. The effectiveness E of the linear regression model given in Equation (1) may be determined using Equation [2]:

E=var(t)*(slope₁²+slope₂²+slope₃²)/(var(a₁)+var(a₂)+var(a₃) [2]

where var(x) represents the variance of a series set forth in Equation [3]:

$${SV}_{i} = \frac{\sqrt{var ( {S}_{i} )}}{mean ( {S}_{i} )} \qquad [ 4 ]$$

where N=10*sampling rate. The segmental variation of the acceleration may be determined by first dividing the acceleration on each axis aᵢ into non-overlapping sub-segments for every 20 points (where the last sub-segment is ignored if it has less than 20 points). The segmental variation SV may then be determined using Equation [4]:

$$var ( x ) = {\sum }_{k = 1}^{N} ( x ( k ) - {mean ( x ( k ) )}^{2} / N - 1 \qquad [ 3 ]$$

where S is the standard deviation within each sub-segment. The feature used by the machine learning model can be the average of the segmental-variation of the three axes: (SV₁+SV₂+SV₃)/3.

The detailed example set forth above is illustrative only, and is not intended to be limiting. In some embodiments, other equations, algorithms, or techniques may be used to identify driving-related or other non-step-related data. In some embodiments, data is compared to signatures of step-related data instead of, or in addition to, signatures of non-step-related data. For example, a generic signature of step-related data may be used, or a user-specific signature may be used. The process 400 may proceed to block 406 if the current input data approximates such a signature.

At block 406, the step counting device 102 can apply one or more filters to remove noise and interference from the data received from the sensor(s) 206, smooth the data, separate out portions of interest, etc. In some embodiments, the filter may be an infinite impulse response (BR) filter. As shown and described here, the smoothed data x(t) for time t may be denoted s_x(t).

At block 408, the step counting device 102 can average input data received over a predetermined or dynamically determined time interval. Illustratively, the time interval may be about 3 seconds, 2 seconds, 1 second, 0.5 seconds, 0.1 seconds, or some other appropriate interval. By averaging the input data over particular time intervals, the averages for two different time intervals (e.g., two successive time intervals) can be compared to determine whether similar data is being received, indicating a consistent state of movement or non-movement. In addition, averaging the input over particular time intervals reduces the effect that anomalous samples have on the step counting process. As shown and described herein, the average input data may be a mean acceleration vector denoted mv, and the time interval may be denoted k. Thus, mv(k) represents the mean acceleration vector for the time interval k.

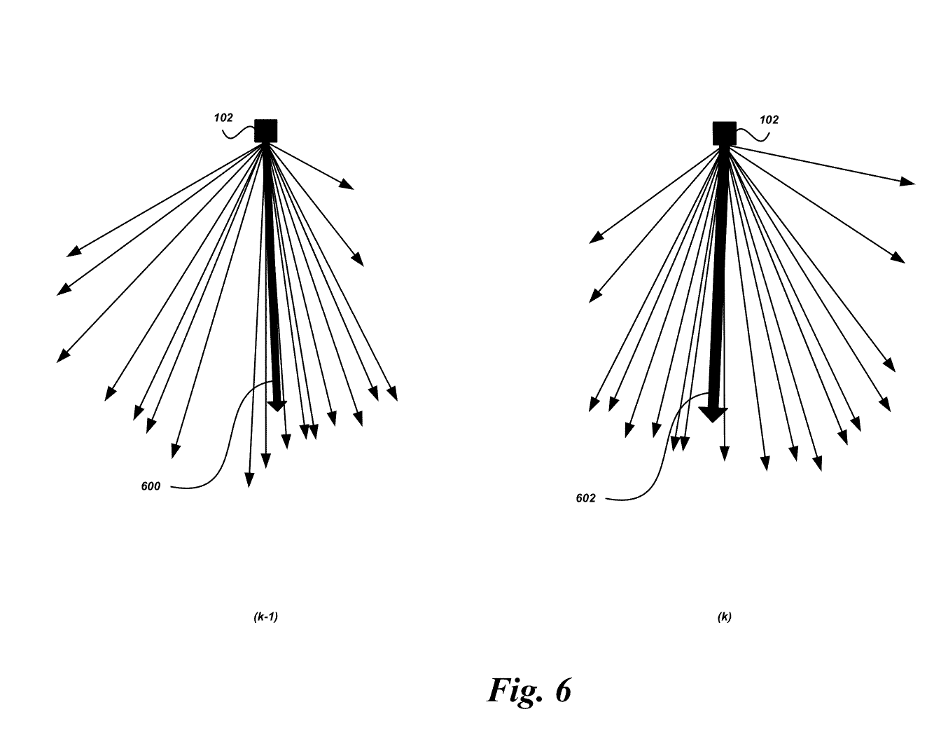

FIG. 6 shows example mean vectors my 600 and 602 calculated for two successive time intervals k−1 and k. As shown, the mean vector mv(k) 602 for a particular time interval k may be calculated as the mean of all data samples obtained during the time interval k. Accordingly, if the sensors 206 are producing data samples at a rate of 26 Hz, then 26 samples may be averaged to calculate the mean vector mv(k) for a the time interval k. Mean vector mv(k−1) 600 corresponds to the mean of all data samples obtained during the prior time interval k−1.

Returning to FIG. 4A, at decision block 410 the step counting device 102 can observe, calculate or otherwise determine the angle between the mean vector mv(k) for the current time interval and the mean vector mv(k−1) for the previous time interval k−1. When a user is walking or running, the orientation of the mean vector my for each short time interval (e.g., 1 second) may not change much. If the angle between the mean vectors for successive time intervals does not fall within a predetermined range or below a threshold, the process 400 can terminate or suppress any subsequent step detection. The process 400 may do so because, e.g., a relatively large angle may be indicative of motion that does not correspond to walking and/or running. As shown in FIG. 6 and described herein, the mean vectors for successive time intervals of sufficient length tend to be similar when a user is walking or running. If the mean vectors are pointing in substantially different directions, that may indicate that the user is doing some other activity, such as waving his arms, eating, etc.

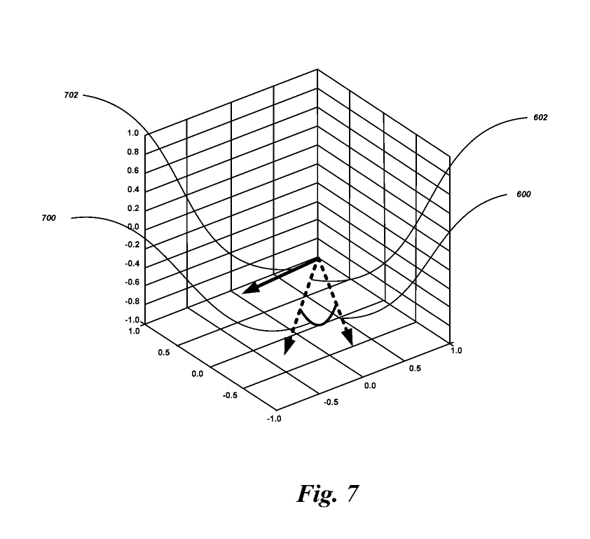

FIG. 7 shows example mean vectors mv(k−1) 600 and mv(k) 602 for successive intervals k−1 and k. When a user is walking or running, the angle 700 between two mean vectors mv(k−1) 600 and mv(k) 602 tend to be less than about 50 degrees. If a user was doing some other activity, the angle may be larger than 50 degrees, such as the angle between mean vector mv(k−1) 600 and mv(x) 702. Upon determining that the angle between the mean vector for the current time interval and the previous time interval is greater than 50 degrees (or some other threshold) the process 400 may terminate.

With continued reference to FIG. 4A, at decision block 412, the step counting device 102 can determine the difference between the current input data (e.g., the smoothed input data s_x(t) from block 406) and the mean vector my. If the difference is greater than some predetermined or dynamically determined threshold, then the process 400 may proceed to block 414; otherwise, the process 400 may terminate. The determination made at decision block 412 can help to ensure that small acceleration changes with respect to the mean acceleration of the current time interval do not form the basis for detection of a step. Illustratively, the threshold may be some percentage or magnitude selected to filter out data that is typically not indicative of a step.

At block 414 the step counting device 102 can average the mean vector for the current time interval mv(k) and the mean vector for the previous time interval mv(k−1) to generate an updated mean vector mv(k). Such averaging further reduces the effect of a single or small number of anomalous samples, and extends the overall time interval from which the mean vector is computed. The updated mean vector mv(k) is then used in detecting peaks of data corresponding to an individual sample or small number thereof. Such peaks may be indicative of a step taken by a user, and therefore the peaks may be used to count steps, as described herein.

At block 416, the step counting device 102 performs peak detection using the updated mean vector mv(k) and the current input sample. FIG. 4 illustrates example operations and processes for detecting acceleration peaks in samples of input data and using those peaks to count steps.

With reference to FIG. 4B, at block 418 the step counting device 102 can project the mean vector mv(k) (as averaged above at block 416) onto the gravity acceleration vector at the current time t to determine the vertical acceleration vector at time t. The use of 3-axis data (e.g., from a 3-axis accelerometer or a gyroscope and accelerometer in combination) allows the step counting device 102 to determine the direction of gravity at time t.

At block 420, the step counting device 102 can determine the first-order derivative of the projected vector determined above. The slope of the derivative at any particular point in time, such as time t, indicates how the vertical acceleration is changing at that time. The derivative (and therefore the slope) can be estimated using vertical acceleration values for adjacent time instances (e.g., using 5 data points). The step counting device 102 can use the change in acceleration over some period of time to identify peaks in the acceleration (e.g., the maximum acceleration in a period of time). The acceleration experienced by the step counting device 102 (and detected by the sensors 206) typically reaches a local peak once during each step taken by the user 100. Thus, by detecting samples in the input data that correspond to the peak acceleration vector in a period of time, individual steps can be detected.

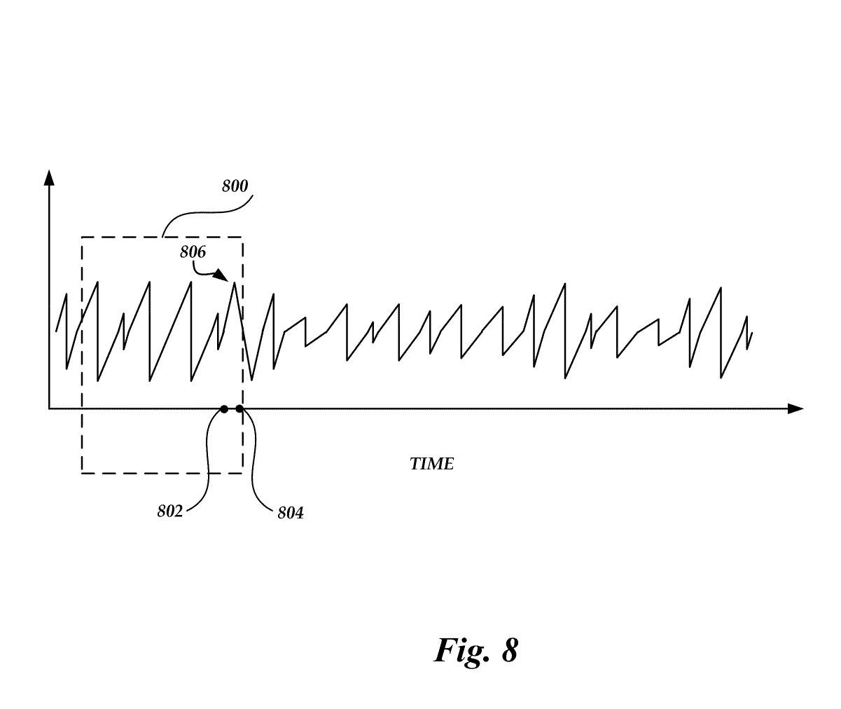

At decision block 422, the step counting device 102 can determine whether the current sample is indicative of a peak for a particular period of time. In some embodiments, the peak may correspond to a local maximum (e.g., a maximum value over some period of time). FIG. 8 shows an illustrative graph of acceleration data over a period of time, include time window 800. Current time t 804 and previous time t−1 802 are indicated on the graph. The step counting device 102 can detect a local maximum 806 at time t−1 802 (or between times t−1 802 and t 804) by determining whether the slope at a point corresponding to time t−1 802 is positive (thereby indicating that the magnitude of acceleration was increasing at time t−1 8-2) and the slope at the current time t 804 is negative (thereby indicating that the magnitude of vertical acceleration is decreasing at time t 804). If the magnitude of acceleration was increasing at time t−1 and is decreasing at time t, then a local maximum has been detected, and the process 400 may continue to decision block 424; otherwise, the process 400 may terminate.

At decision block 424, the step counting device 102 can determine whether the number of peaks identified for a predetermined or dynamically determined window of time meets or exceeds some threshold. When a user is walking or running, the user typically takes several steps in succession, rather than individual steps followed by periods of no activity. By implementing a threshold number of steps for a window of time (such as window 800 shown in FIG. 8), the step counting device 102 can avoid false positives in cases where a small number of peaks (or only 1 peak) have been observed in a period of time in which a larger number of peaks would be expected if the user were actually walking or running. In some embodiments, the step counting device 102 may operate in at least two states: a first state in which the step counting device 102 is waiting until a threshold number of steps have been taken in a window of time before counting the steps, and a second state in which the threshold has been met, and the step counting device 102 counts steps for each peak identified above. If the threshold has been met, the process 400 can proceed to block 426; otherwise, the process 400 can proceed to block 428.

At block 426, the step counting device 102 can increment a step counter or otherwise record detection of a step. The process 400 may then terminate. In some embodiments, information can be stored in volatile memory (e.g., random access memory) or in non-volatile memory (e.g., flash memory) integrated with or otherwise in communication with the step counting device 102. In some embodiments, a notification or other data regarding the detected step or the current sum of the step counter can be transmitted to another computing device, presented to the user, or the like. In further embodiments, detection of a step can trigger other processes, such as execution of one or more applications or an analysis of the number of steps.

At block 428, the step counting device 102 can increment a threshold counter (also referred to herein as a “gatekeeper counter”). The gatekeeper counter can be used to track the number of peaks, identified above, for the current window of time. The gatekeeper counter can be inspected at decision block 424, above, to determine whether the number of peaks identified for the window has met the threshold. In some embodiments, rather than inspecting the gatekeeper counter, a flag or other state indicator may be set at block 428 in response to identifying a threshold number of peaks. The step counting device 102 can then check the flag at block 426 in order to determine whether to proceed to block 426 or 428.

At block 430, the step counting device 102 can again determine whether the threshold number of peaks in the current window of time has been reached, similar to the determination made above at block 424. If a threshold has been reached, the process 400 can continue to block 432; otherwise, the process 400 may terminate.

At block 432, the gatekeeper counter can be added to the step counter. As described herein, the gatekeeper counter may be incremented in cases where a threshold number of steps have not yet been recorded (and therefore the step counter is not incremented). However, once the threshold has been reached, the step counting device 102 can infer that the previously detected peaks were indeed steps and should be counted along with all subsequently detected steps in the current window. Accordingly, the step counting device 102 can add the gatekeeper counter to the step counter and reset the gatekeeper counter for use in the next time window.

In some embodiments, the activity determination device 102 can be used to determine an activity of the wearer (e.g., to determine whether the wearer is walking, running, driving, sleeping, etc.). The processor 204 can analyze data from the one or more sensors 206 (e.g., one or more accelerometers) to determine a user activity. The processor 204 can analyze the sensor data for intervals of time, and can determine whether the sensor data for each interval of time corresponds to a particular user activity. If a sufficient number of the intervals correspond to the same user activity in a specified time, the processor can determine that the entire specified time corresponds to the activity (e.g., even if the data for some number of the intervals does not correspond to the activity).

By way of example, the processor 204 can analyze data from the one or more sensors 206 (e.g., one or more accelerometers) to determine whether the wearer is driving. The processor 204 can analyze the sensor data in 10 second intervals to determine whether the sensor data for each interval corresponds to a driving activity (e.g., as described above). After 60 seconds of data, if the sensor data for 3 or more of the 6 time intervals corresponds to the driving activity, the processor 204 can determine that the wearer was driving during the measured 60 seconds (e.g., even though 1, or 2, or 3 of the 10-second time intervals may not have sensor data that corresponds to driving). Accordingly, the processor 204 can determine that the wearer is driving even if the wearer makes movements from time to time that do not fit the profile of the driving activity (e.g., moving hands to take a drink or to stretch). Other time values can be used in other embodiments.

By way of another example, the processor 204 can analyze data from the one or more sensors 206 (e.g., one or more accelerometers) to determine whether the wearer is sleeping. The processor 204 can analyze the sensor data in 1 minutes intervals or sections to determine whether the sensor data for each 1-minute interval corresponds to the sleeping activity. After 90 minutes, if a sufficient number of the 1-minute intervals corresponded to the sleeping activity, the processor 204 can determine that the user was sleeping for the 90 minutes that were measured (e.g., even if some of the intervals have sensor data that does not correspond to sleeping). Accordingly, the processor 204 can determine that the wearer was sleeping even if the wearer makes movements from time to time that do not fit the profile of the sleeping activity (e.g., rolling over). In some embodiments, if the wearer were to get up to use the bathroom or get a drink and then return to bed, only a small number of the 1-minute time intervals would not match the sleeping activity profile, and the processor 204 can determine that the wearer was sleeping during those time. The processor 204 can calculate a sleep score for the time period in which the wearer was determined to be sleeping. The sleep score can depend at least in part on the number of intervals during the sleep time period that have sensor data that does not fit the sleep activity profile. Thus, if a user were to toss and turn during sleep or get up to use the bathroom, those actions can increase the number of non-sleep intervals, which can reduce the sleep score.

### Terminology

Depending on the embodiment, certain acts, events, or functions of any of the processes or algorithms described herein can be performed in a different sequence, can be added, merged, or left out altogether (e.g., not all described operations or events are necessary for the practice of the algorithm). Moreover, in certain embodiments, operations or events can be performed concurrently, e.g., through multi-threaded processing, interrupt processing, or multiple processors or processor cores or on other parallel architectures, rather than sequentially.

The various illustrative logical blocks, modules, routines, and algorithm steps described in connection with the embodiments disclosed herein can be implemented as electronic hardware, or as a combination of electronic hardware and executable software. To clearly illustrate this interchangeability, various illustrative components, blocks, modules, and steps have been described above generally in terms of their functionality. Whether such functionality is implemented as hardware, or as software that runs on hardware, depends upon the particular application and design constraints imposed on the overall system. The described functionality can be implemented in varying ways for each particular application, but such implementation decisions should not be interpreted as causing a departure from the scope of the disclosure.

Moreover, the various illustrative logical blocks and modules described in connection with the embodiments disclosed herein can be implemented or performed by a machine, such as a general purpose processor device, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general purpose processor device can be a microprocessor, but in the alternative, the processor device can be a controller, microcontroller, or state machine, combinations of the same, or the like. A processor device can include electrical circuitry configured to process computer-executable instructions. In another embodiment, a processor device includes an FPGA or other programmable device that performs logic operations without processing computer-executable instructions. A processor device can also be implemented as a combination of computing devices, e.g., a combination of a DSP and a microprocessor, a plurality of microprocessors, one or more microprocessors in conjunction with a DSP core, or any other such configuration. Although described herein primarily with respect to digital technology, a processor device may also include primarily analog components. For example, some or all of the signal processing algorithms described herein may be implemented in analog circuitry or mixed analog and digital circuitry. A computing environment can include any type of computer system, including, but not limited to, a computer system based on a microprocessor, a mainframe computer, a digital signal processor, a portable computing device, a device controller, or a computational engine within an appliance, to name a few.

The elements of a method, process, routine, or algorithm described in connection with the embodiments disclosed herein can be embodied directly in hardware, in a software module executed by a processor device, or in a combination of the two. A software module can reside in RAM memory, flash memory, ROM memory, EPROM memory, EEPROM memory, registers, hard disk, a removable disk, a CD-ROM, or any other form of a non-transitory computer-readable storage medium. An exemplary storage medium can be coupled to the processor device such that the processor device can read information from, and write information to, the storage medium. In the alternative, the storage medium can be integral to the processor device. The processor device and the storage medium can reside in an ASIC. The ASIC can reside in a user terminal. In the alternative, the processor device and the storage medium can reside as discrete components in a user terminal.

Conditional language used herein, such as, among others, “can,” “could,” “might,” “may,” “e.g.,” and the like, unless specifically stated otherwise, or otherwise understood within the context as used, is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without other input or prompting, whether these features, elements and/or steps are included or are to be performed in any particular embodiment. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list.

Disjunctive language such as the phrase “at least one of X, Y, Z,” unless specifically stated otherwise, is otherwise understood with the context as used in general to present that an item, term, etc., may be either X, Y, or Z, or any combination thereof (e.g., X, Y, and/or Z). Thus, such disjunctive language is not generally intended to, and should not, imply that certain embodiments require at least one of X, at least one of Y, or at least one of Z to each be present.

Unless otherwise explicitly stated, articles such as “a” or “an” should generally be interpreted to include one or more described items. Accordingly, phrases such as “a device configured to” are intended to include one or more recited devices. Such one or more recited devices can also be collectively configured to carry out the stated recitations. For example, “a processor configured to carry out recitations A, B and C” can include a first processor configured to carry out recitation A working in conjunction with a second processor configured to carry out recitations B and C.

While the above detailed description has shown, described, and pointed out novel features as applied to various embodiments, it can be understood that various omissions, substitutions, and changes in the form and details of the devices or algorithms illustrated can be made without departing from the spirit of the disclosure. As can be recognized, certain embodiments described herein can be embodied within a form that does not provide all of the features and benefits set forth herein, as some features can be used or practiced separately from others. The scope of certain embodiments disclosed herein is indicated by the appended claims rather than by the foregoing description. All changes which come within the meaning and range of equivalency of the claims are to be embraced within their scope.

What is claimed is:

1. A device comprising:
one or more sensors configured to detect acceleration;
one or more physical computer processors configured to at least:
obtain acceleration data from the one or more sensors for a time interval;
determine that the acceleration data is not indicative of a non-step-related activity;
average the acceleration data for the time interval to obtain a mean acceleration vector;
project the mean acceleration vector on a gravity acceleration vector to obtain a projection;
identify a peak using a derivative based at least partly on the projection; and
determine that a user of the device has taken a step based at least partly on the identified peak.

2. The device of claim 1, wherein the one or more computer processors are configured to determine that the acceleration data is not indicative of a non-step-related activity by extracting one or more features from the acceleration data and analyzing the features to determine whether they correspond to features of data associated with a non-step-related activity.

3. The device of claim 1, wherein the non-step-related activity comprises driving, eating, brushing teeth, or sleeping.

4. The device of claim 1, wherein the one or more computer processors are configured to determine that the acceleration data is not indicative of a non-step-related activity by analyzing the acceleration data with respect to data associated with mechanical vibrations.

5. The device of claim 1, wherein the one or more computer processors are further configured to determine whether a number of peaks identified in a window of time exceeds a threshold.

6. The device of claim 1, wherein the one or more sensors comprises a 3-axis accelerometer.

7. The device of claim 1, wherein the one or more processors are further configured to obtain data from a plurality of sensors.

8. The device of claim 7, wherein the plurality of sensors comprises an accelerometer and a gyroscope.

9. The device of claim 1, wherein the one or more processors are configured to at least:
identify a plurality of steps during a time window;
determine that the user is performing a stepping activity when the plurality of steps during the time window are above a threshold number; and
determine that the user is not performing a stepping activity when the plurality of steps during the time window are not above the threshold number.

10. Non-transitory computer-readable storage storing executable instructions that cause one or more computer processors to perform a process comprising:
obtaining acceleration data for a first time interval;
determining that the acceleration data is indicative of an active state of a user;
averaging the acceleration data for the first time interval to obtain a mean acceleration vector;
projecting the mean acceleration vector on a gravity acceleration vector to obtain a projection;
identifying a peak using a derivative based at least partly on the projection; and
determining that the user has taken a step based at least partly on the identified peak.

11. The non-transitory computer-readable medium of claim 10, wherein determining that the acceleration data is indicative of an active state of a user comprises using a classification model trained to detect a signature of user activity in acceleration data.

12. The non-transitory computer-readable medium of claim 10, wherein the process further comprises:
obtaining acceleration data for a second time interval;
determining that the acceleration data is indicative of an inactive state of a user;
changing a rate at which acceleration data is sampled for a third time interval.

13. The non-transitory computer-readable medium of claim 12, wherein the process further comprises:
obtaining acceleration data for the third time interval;
determining that a movement magnitude represented by the acceleration data fails to exceed an idle state threshold; and
determining that the user is sleeping based at least partly on the determination that the movement magnitude fails to exceed the idle state threshold.

14. The non-transitory computer-readable medium of claim 12, wherein the process further comprises:
obtaining acceleration data for the third time interval;
determining that a movement magnitude represented by the acceleration data exceeds an idle state threshold; and
determining that the user is not sleeping based at least partly on the determination that the movement magnitude exceeds the idle state threshold.

15. The non-transitory computer-readable medium of claim 12, wherein the process further comprises:
obtaining acceleration data for the third time interval;
determining that a movement magnitude represented by the acceleration data fails to exceed a sleeping state threshold; and
determining that the acceleration for the third time interval is indicative of non-use.

16. A system comprising:
one or more sensors configured to detect acceleration;
one or more computer processors configured to at least:
obtain acceleration data from the one or more sensors for a first time interval;
determine that the acceleration data for the first time interval is indicative of an active state of a user;
obtain acceleration data from the one or more sensors for a second time interval;
determine that the acceleration data for the second time interval is indicative of an inactive state of a user; and
change a rate at which acceleration data is sampled for a third time interval.

17. The system of claim 16, wherein the one or more computer processors are configured to determine that the acceleration data is indicative of an active state of a user comprises using a classification model trained to detect a signature of user activity in acceleration data.

18. The system of claim 16, wherein the one or more computer processors are configured to at least:
average the acceleration data for the first time interval to obtain a mean acceleration vector;
project the mean acceleration vector on a gravity acceleration vector to obtain a projection;
identify a peak using a derivative based at least partly on the projection; and
determine that the user has taken a step based at least partly on the identified peak.

19. The system of claim 16, wherein the one or more computer processors are further configured to at least:
obtain acceleration data for the third time interval;
determine that a movement magnitude represented by the acceleration data fails to exceed an idle state threshold; and
determine that the user is sleeping based at least partly on the determination that the movement magnitude fails to exceed the idle state threshold.

20. The system of claim 16, wherein the one or more computer processors are further configured to at least:
obtain acceleration data for the third time interval;
determine that a movement magnitude represented by the acceleration data exceeds an idle state threshold; and
determine that the user is not sleeping based at least partly on the determination that the movement magnitude exceeds the idle state threshold.

21. The system of claim 16, wherein the one or more computer processors are further configured to at least:
obtain acceleration data for the third time interval;
determine that a movement magnitude represented by the acceleration data fails to exceed a sleeping state threshold; and
determine that the acceleration for the third time interval is indicative of non-use.

22. The system of claim 16, wherein the one or more processors are configured to at least:
identify a plurality of steps during a time window;
determine that the user is performing a stepping activity when the plurality of steps during the time window are above a threshold number; and
determine that the user is not performing a stepping activity when the plurality of steps during the time window are not above the threshold number.
